(19) United States
(12) Patent Application Publication
(10) Pub. No.: US 2023/0240846 A1
(43) Pub. Date: Aug. 3, 2023

(54) Devices, Systems, and Methods for a Heart-Valve Annulus Reinforcer

(71) Applicant: ReValve Solutions Inc., Bainbridge Island, WA (US)

(72) Inventors: Julie Logan Sands, Bainbridge Island, WA (US); Kenneth Eugene Perry, Bainbridge Island, WA (US); Edward Ian Wulfman, Bainbridge Island, WA (US); Philip Andrew Ahrens, Bainbridge Island, WA (US)

(73) Assignee: ReValve Solutions Inc., Bainbridge Island, WA (US)

(21) Appl. No.: 18/012,970

(22) PCT Filed: Jun. 28, 2021
(86) PCT No.: PCT/US2021/039451

§ 371 Date: Dec. 26, 2022

Related U.S. Application Data
(60) Provisional application No. 63/044,937, filed on Jun. 26, 2020.

(51) Int. Cl. A61F 2/24

(52) U.S. Cl. CPC A61F 2/2445; A61F 2/2466; A61F 2220/0016; A61F 2230/005; A61F 2230/0069; A61B 2018/00369

(57) ABSTRACT

Devices, systems, and methods for heart-valve annulus repair techniques, comprising a reinforcing device with one or both of a heating mechanism or clamping mechanism. The heating mechanism may function by heating a localized portion of the annulus so as to reduce the circumference of the annulus and/or increase the stiffness of the tissue. The clamping mechanism may function by gathering or pinching an area of tissue to then reduce the circumference of the annulus and/or increase the stiffness of the tissue.

### CROSS-REFERENCE TO RELATED APPLICATIONS

0001. This application claims benefit to U.S. Provisional Application No. 63/044,937, filed on Jun. 26, 2020, titled Devices, Systems, and Methods for a Mitral Annulus Reinforcer, the contents of which are incorporated by reference herein as though set forth in their entirety, and to which priority and benefit are claimed.

### FIELD OF USE

The present disclosure relates generally to organ-tissue modification technology and more specifically to reinforcement devices, systems, and methods for modifying the tissue in a localized area of an organ, such as in reducing the distension of a valve annulus.

### BACKGROUND

Valve repair procedures are regularly performed to treat diseases of the valves of the heart. In heart valve regurgitation, a type of heart valve pathology, the leaflets (flaps) of the mitral valve do not close sufficiently, causing blood to flow backward and degrade the efficiency of the valve. If regurgitation is significant, blood may not flow efficiently through the heart or rest of the body, leading to a number of mild and severe symptoms. When regurgitation is due to the dilation of the annulus—the ring around the valve—annuloplasty is one approach in repair surgery. Annuloplasty is a surgical procedure for tightening or reinforcing the annulus and is performed by using a catheter, rings and clips, or other devices. The procedure consists of measuring the size of the annulus and sewing a band to the annulus around the valve.

Unfortunately, the current procedure and devices for treatment of valve regurgitation pose limitations and risks. For example, a percutaneous leaflet procedure focuses on stabilizing the leaflet (edge-to-edge procedure) but does not address the dilation of the valve therefore a significant reduction or complete elimination of regurgitation cannot be achieved.

Thus, what is needed are devices, systems, and methods for a fast and simple method for reducing dilation of the valve annulus and/or stiffening the annulus in order to further reduce valve regurgitation. The individual elements for such devices, systems, and methods should not interfere with the overall motion of the mitral anulus as does a full-length, or partial-length annuloplasty ring. In addition, these elements must be precisely and independently deployed to effectively provide the desired effect while avoiding critical structures; for example, the AV node or central fibruous body when treating the tricuspid valve. The treatment should also enable use in conjunction with other valve repair techniques.

This disclosure is applicable to any valve or any area where it is desired to achieve a localized change, alone or as part of a coordinated, multi-device procedure, in tissue topology or behavior.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to devices, systems, and methods for heart-valve annulus repair techniques. Such techniques comprise a reinforcing device with one or both of a heating mechanism or clamping mechanism. The heating mechanism may function by heating a localized portion of the annulus so as to reduce the circumference of the annulus and/or increase the stiffness of the tissue. The clamping mechanism may function by gathering or pinching an area of tissue to then reduce the circumference of the annulus and/or increase the stiffness of the tissue.

Providing multiple independent and potentially unconnected elements to reduce distension of the valve annulus, as done here, is a novel approach for treatment of heart-valve regurgitation. And it is a novel approach to provide a solution for augmentation of previous repair approaches. Such a disclosure is an improvement over existing techniques because the individual elements are not overstressed by the motion of the heart and because the present disclosure does not interfere with the native functioning of the annulus yet still provides annular reduction and stiffening. The present disclosure thus allows for the ultimate in flexibility and custom tailoring of enhancement therapy.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1A generally illustrates an embodiment of an annulus repair system as disclosed herein.

FIG. 1B generally illustrates an embodiment an annulus repair system as disclosed herein.

FIG. 1C generally illustrates an embodiment of an annulus repair system as disclosed herein.

FIG. 2A generally illustrates an embodiment of an annulus repair system as disclosed herein.

FIG. 2B generally illustrates an embodiment of a mitral—annulus repair system as disclosed herein.

FIG. 3 generally illustrates an embodiment of a reinforcing device of an annulus repair system as disclosed herein.

FIG. 4 generally illustrates an embodiment of a reinforcing device of an annulus repair system as disclosed herein.

FIG. 5 generally illustrates an embodiment of a reinforcing device of an annulus repair system as disclosed herein.

FIG. 6 generally illustrates an embodiment of an annulus repair system as disclosed herein.

FIG. 7 generally illustrates an embodiment of an annulus repair system as disclosed herein.

FIG. 8 generally illustrates an embodiment of an annulus repair system as disclosed herein.

FIG. 9A-9C generally illustrates an embodiment of an annulus repair system as disclosed herein.

FIG. 10A-10D generally illustrates an embodiment of an annulus repair system as disclosed herein.

FIG. 11A-11C generally illustrates an embodiment of an annulus repair system as disclosed herein.

FIG. 12 generally illustrates an embodiment of a reinforcing device of an annulus repair system as disclosed herein.

FIG. 13 generally illustrates an embodiment of a reinforcing device of an annulus repair system as disclosed herein.

FIG. 14 generally illustrates an embodiment of a reinforcing device of an annulus repair system as disclosed herein.

FIG. 15 generally illustrates an embodiment of a reinforcing device of an annulus repair system as disclosed herein.

FIGS. 16A-16B generally illustrates an embodiment of a reinforcing device of an annulus repair system as disclosed herein.

FIGS. 17A-17B generally illustrates an embodiment of a reinforcing device of an annulus repair system as disclosed herein.

FIGS. 18-20 generally illustrates an embodiment of an annulus repair system as disclosed herein.

### DETAILED DESCRIPTION OF EMBODIMENTS

Before the present devices, systems, and methods are disclosed and described, it is to be understood that the disclosure is not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

Disclosed herein is a system for the repair and reinforcement of the annulus of a heart valve (“Reinforcer”). In one embodiment, the Reinforcer comprises a deployment system for the deployment of a reinforcing device that reinforces the tissue of the valve annulus. The reinforcing device may comprise either a heating mechanism or a clamping mechanism.

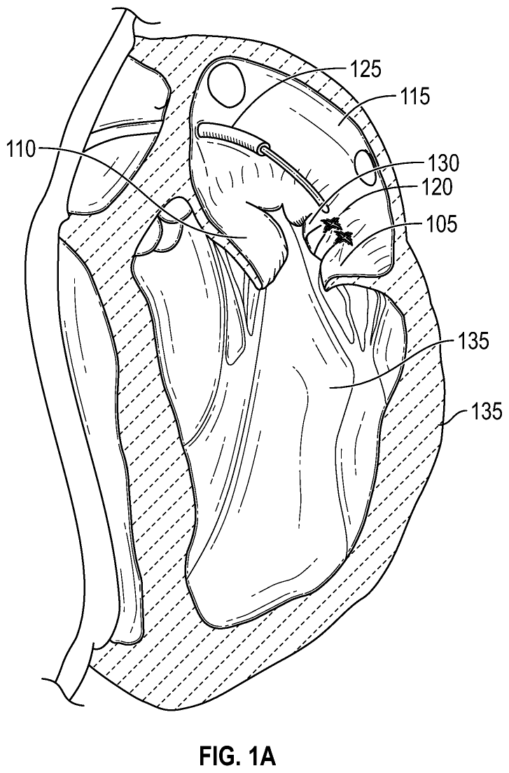

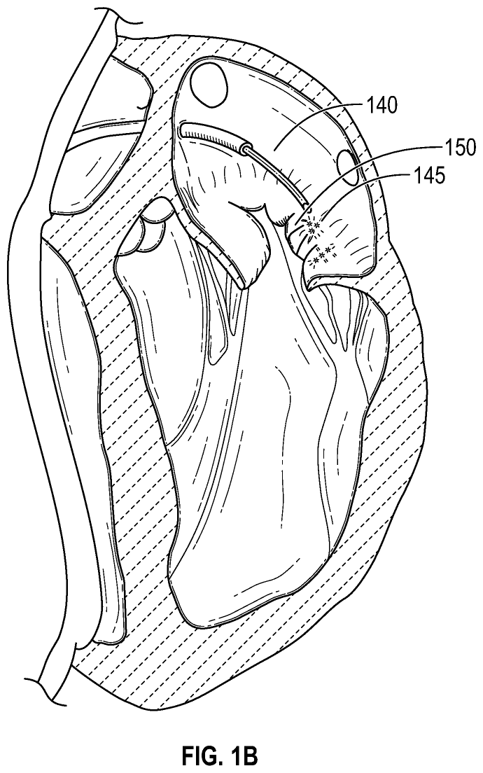

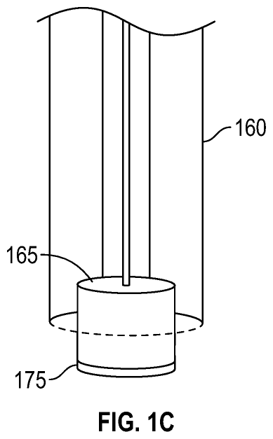

FIG. 1A generally illustrates an embodiment of an annulus repair system as disclosed herein. FIG. 1A discloses a mitral valve, a part of the structural anatomy of the left heart consisting of the left atrium 115, left ventrical 135, and miocardium 135. The mitral valve further comprises a posterior leaflet 105 and an anterior leaflet 110, with a malformation of the posterior leaflet 105 that causes dysfunction.

In a preferred embodiment, a catheter 125 is delivered via the septum of the left atrial transseptal, though the catheter 125 may be delivered via other approaches know in the art, such as trans-aortically. As shown in FIG. 1A, contraction clips 120 (termed here “reinforcing devices” along with the heating mechanism) of the Reinforcer are delivered via the catheter 125 and are implanted in the posterior annulus and/or leaflet 105. When implanted, the reinforcing devices 120 modify the geometry of the annulus and/or leaflet by contracting the tissue 130, thus changing the annulus and/or leaflet in a therapeutic manner.

FIG. 1B generally illustrates an embodiment of an annulus repair system as disclosed herein. As shown in FIG. 1B, an embodiment of the Reinforcer comprises a laser catheter 140, wherein the laser catheter 140 delivers energy to the posterior annulus and/or leaflet 105 in a “spot weld” manner 145, such that the tissue is modified and shrinks or tightens 150, thus modifying the annulus and/or leaflet in a therapeutic manner to improve leaflet function. In one embodiment, the temperature may be a set temperature and non-adjustable. In another embodiment, the on/off is controlled through a switch or button on the delivery catheter handle.

FIG. 1C generally illustrates an embodiment of an annulus repair system as disclosed herein. FIG. 1C discloses a reinforcing device comprising a heating mechanism. As shown in FIG. 1C, the Reinforcer may comprise a catheter 160 and a pusher platform 165 for the deployment of a heating mechanism 175. In this embodiment, the device may be deployed by the use of the pusher platform 165, wherein the heating mechanism 175 is pressed up against the tissue so as to shrink and tighten the localized portion. In other embodiments, after initial application of the heating mechanism 175, the localized area may be somewhat loosened and expanded, yet the skin is still tighter than before and the heating mechanism 175 may be reapplied until the desired reinforcement is achieved. The heat serves to shrink and pull in the skin. Heating mechanisms may be heated via electricity, electromagnetism, or other methods that produce heat.

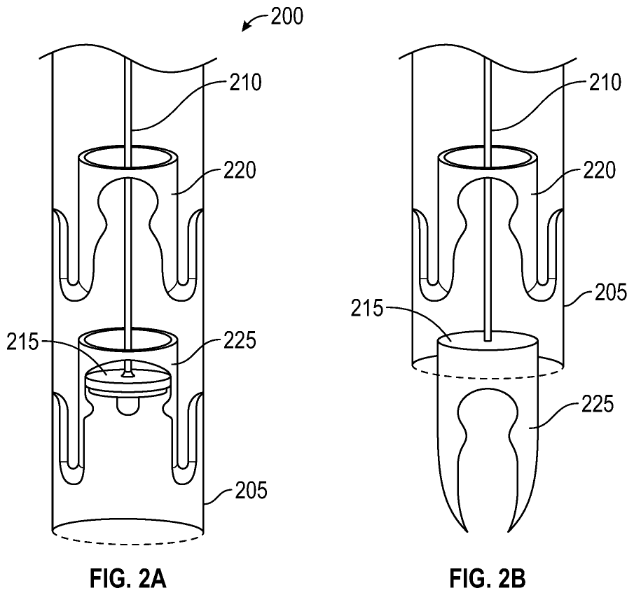

FIG. 2A generally illustrates an embodiment of an annulus repair system as disclosed herein. As shown in FIG. 2A, the Reinforcer 200 may comprise a deployment system, such as the combination of a catheter 205, a coaxial pusher 210 and a pusher platform 215. The Reinforcer may further comprise two reinforcing devices 220, 225, which are loaded into the catheter 205. The pusher platform 215 provides a platform for applying pressure onto to push a device 220, 225 out of the catheter 205. The pusher platform 215 slides along the inside diameter of the device 220, 225 to be deployed until the device 220, 225 is retracted enough to enlarge under its own spring-loaded force. Deployment systems like the catheter 205 and pusher platform 215 combination enable the delivery of multiple devices to very precise locations. And they may also be capable of removing devices, such as in the event of misplacement or changing indications.

FIG. 2B generally illustrates an embodiment of an annulus repair system as disclosed herein. FIG. 2B discloses an embodiment of a reinforcing device 225 being deployed. The process for deploying reinforcing devices may be repeated by retracting the pusher platform 215 through the next device to be deployed. In these embodiments, the catheter 205 permits rapid and accurate delivery of multiple reinforcing devices.

In one embodiment, reinforcing devices are loaded into a delivery catheter with the scooped tips bent 180 degrees. Multiple reinforcing devices may be used for a patient such that a catheter is loaded with the devices capable of rapidly and accurately delivering them percutaneously. Each device is individually positioned against the tissue at the desired location and then deployed so that the scooped tips grab the tissue, pull it together, and anchor it. The scooped tips may thus comprise penetrating elements to secure to the tissue and compress it so as to enable reduction of circumference or stiffening of the annulus. For example, the Reinforcer may reduce mitral regurge by 1 grade or a fraction of a grade (e.g. 3 to 2.5). And the deployment system may serve to remove reinforcing devices. For example, a pinching action on the proximal end of a device may cause the release of the tissue from the device.

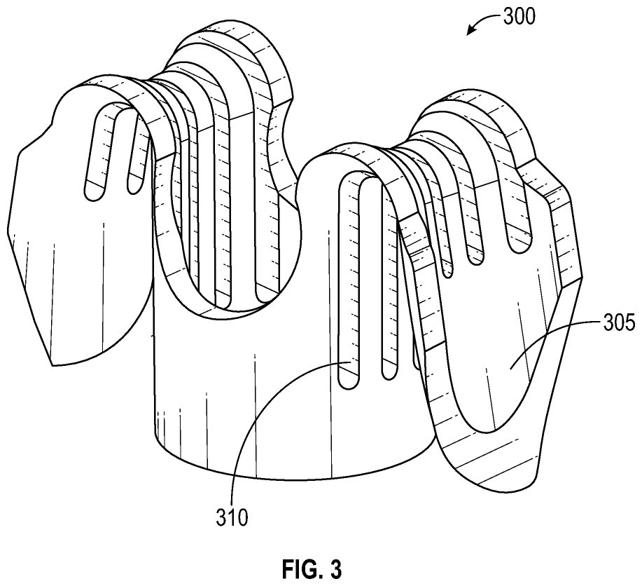

FIG. 3 generally illustrates an embodiment of a reinforcing device of an annulus repair system as disclosed herein. As shown in FIG. 3, a reinforcing device 300 may be a hollow cylindrical structure with a top end and a bottom end, wherein both the top end and the bottom end are open. The reinforcing device 300 may further have a plurality of arms extending down from the top end, wherein the plurality of arms end with a penetrating element and wherein the plurality of arms are configured to bend out away from the device.

The reinforcing device 300 in FIG. 3 is in the catheter-loaded position, with scooped tips 305 that are used for penetrating and attaching to the skin. This is the position that reinforcing devices are in prior to being deployed. Reinforcing devices may also comprise stress-relieving slots 310 and a co-axial control/release mechanism.

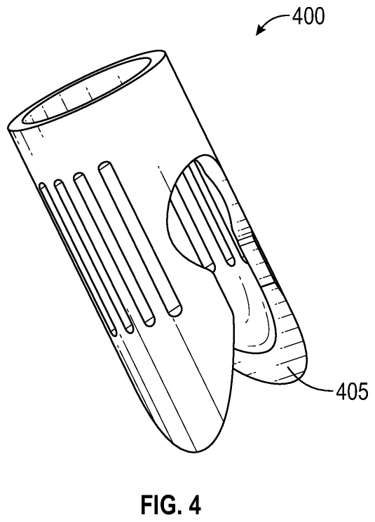

FIG. 4 generally illustrates an embodiment of a reinforcing device of an annulus repair system as disclosed herein. The reinforcing device 400 in FIG. 4 is in the deployed configuration, with the scooped tips extended 405.

In one embodiment, reinforcing devices with clamping mechanisms are manufactured through the laser cutting and electropolishing of nitinol tubes. Alternate processes include but are not limited to EDM and chemical etching. The penetration elements and overall design of the scooped tips may be shape-set to further enhance their effectiveness. Other embodiments may comprise varying stress-relief elements, different scooped tip designs, and different coaxial control elements/features. Embodiments may also comprise reinforcing devices with coatings that can elicit responses through drug delivery or sensors attached to them. Anchor lines may also be attached to a reinforcing device to create anchoring of a tethered structure, such as to create a replacement for chordae to support leaflet function or to anchor a device such as a replacement valve. Overall, reinforcing devices need be durable and biocompatible and able to withstand loading into the catheter and subsequent deployment. In another embodiment, the Reinforcer has the capability to remove and/or replace devices in its deployment system.

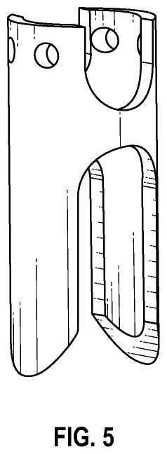

FIG. 5 generally illustrates an embodiment of a reinforcing device of an annulus repair system as disclosed herein. As shown in FIG. 5, a reinforcing device may comprise straight penetration elements and a hinge (like an old-style clothes pin). The design may also have scooped tips and both an opened and closed configuration. The end with the small holes would thus move closer together when in the open configuration. It could be self-actuating as shown, or it could include an additional element to force the top end (the end with the small holes) apart when activated to close. For example, a solid object could initially be positioned inside the tube and below the small holes and then moved between the small holes to force the device to close. This may create a very rigid and forceful actuation of the clip.

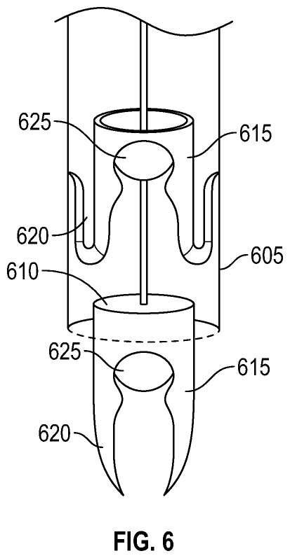

FIG. 6 generally illustrates an embodiment of an annulus repair system as disclosed herein. FIG. 6 discloses a reinforcing device with both a clamping and heating mechanism. As shown in FIG. 6, the Reinforcer may comprise a catheter 605 and a pusher platform 610 for the deployment of a reinforcing device 615 with a clamping mechanism 620 and a heating mechanism 625. In this embodiment, the device 615 may be deployed as disclosed in FIGS. 1-5 with the addition that the heating mechanism 625 may provide additional support for reducing dilation of the tissue or stiffening the tissue. In one embodiment, once the device 615 has penetrated and attached to the skin, the heating mechanism 625 may be engaged to provide heat to the localized area.

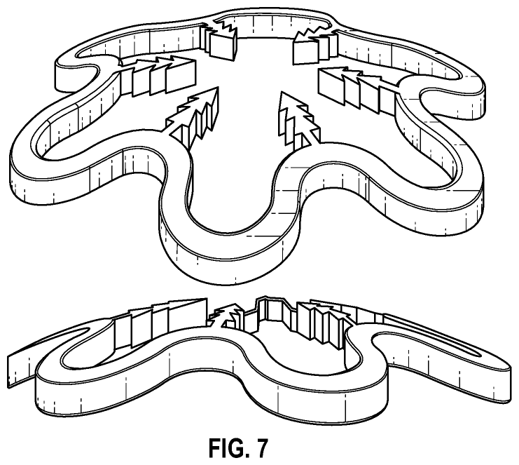

FIG. 7 generally illustrates an embodiment of an annulus repair system as disclosed herein. As shown in FIG. 7, a reinforcing device may be circular or somewhat star-shaped, comprising inward-pointing elements designed and configured to engage the tissue and pull inward when deployed/activated. These inward-pointing elements possess features that facilitate the engagement and secure containment of the tissue. In one embodiment, a texture/intentional disruption/pattern may be featured on the cutting edges of the elements to reduce the force it takes for the elements to penetrate the tissue (much in the same way that porcupine quills reduce the force necessary to achieve skin penetration and retain that penetration through their natural geometry).

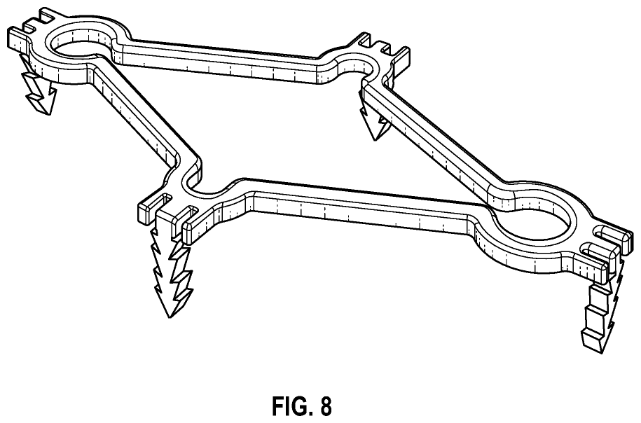

FIG. 8 generally illustrates an embodiment of an annulus repair system as disclosed herein. As shown in FIG. 8, a reinforcing device may employ the features described above regarding the tissue-penetrating elements, further comprising a “diamond” or other non-uniform shape. In this embodiment, the device uses anchors positioned along the midline of the device, wherein the pulling or gathering of tissue in perpendicular directions may be done with specific bias.

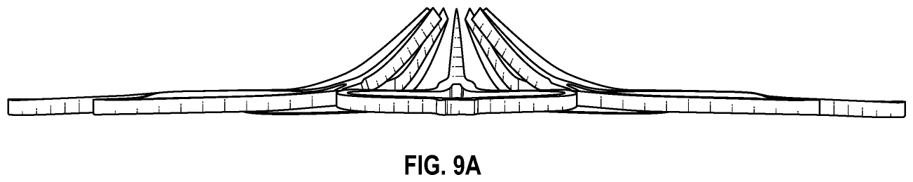

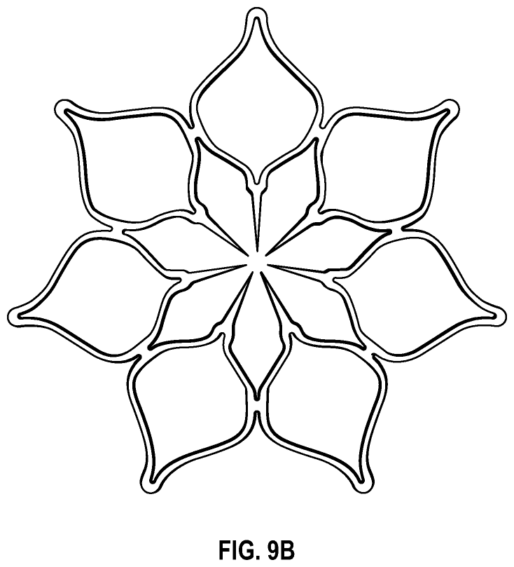

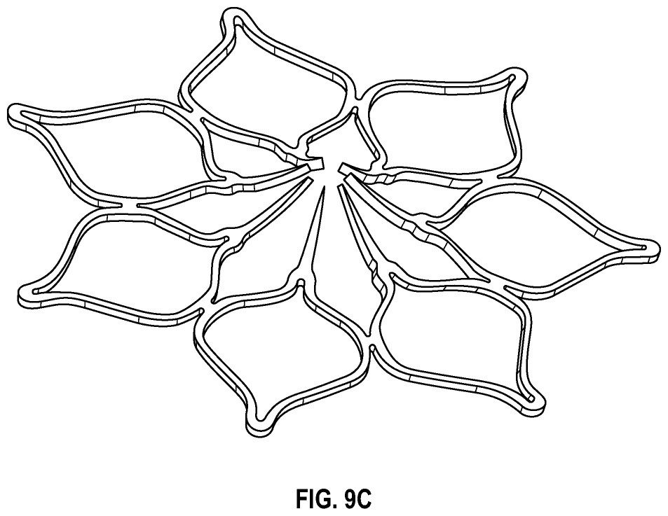

FIG. 9A-9C generally illustrates an embodiment of an annulus repair system as disclosed herein. As shown in FIGS. 9A-9C, a reinforcing device may have radial symmetry, and be circular or somewhat star-shaped, comprising inward-pointing elements designed and configured to engage the tissue and pull inward when deployed/activated. These inward-pointing elements possess features that facilitate the engagement and secure containment of the tissue. In one embodiment, a texture/intentional disruption/pattern may be featured on the cutting edges of the elements to reduce the force it takes for the elements to penetrate the tissue.

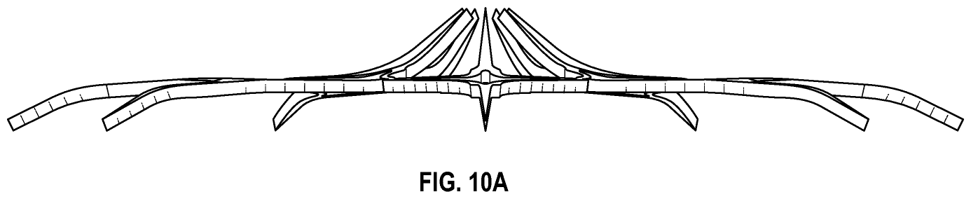

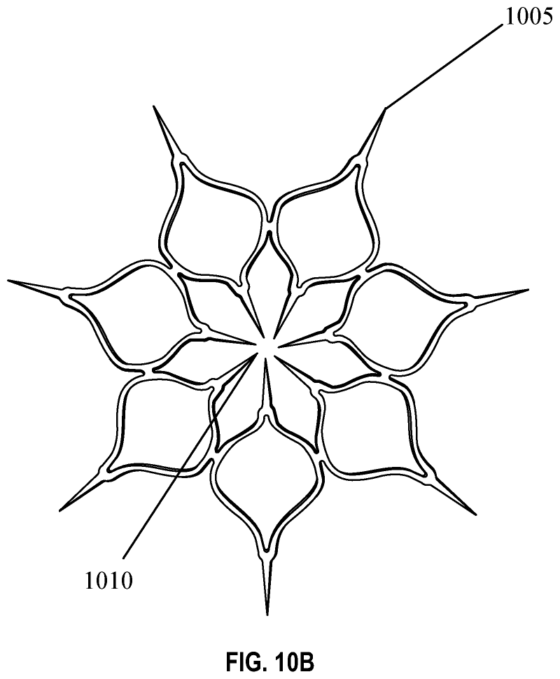

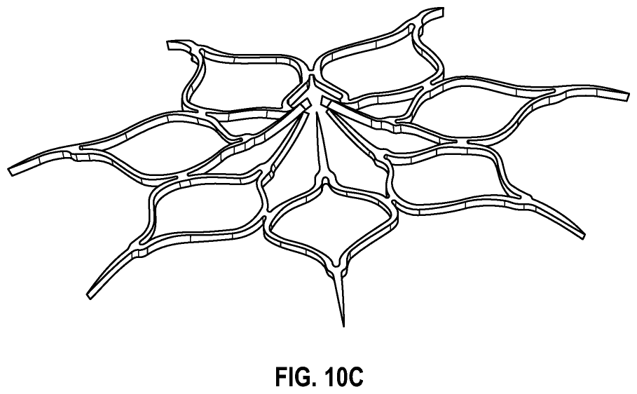

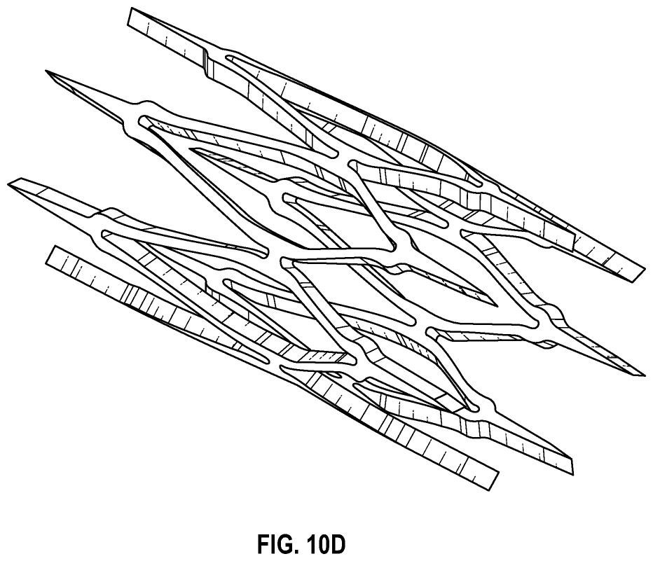

FIG. 10A-10D generally illustrates an embodiment of an annulus repair system as disclosed herein. Specifically, FIG. 10A-10C illustrate an embodiment of the geometry and final shape of reinforcing device when deployed. Also shown in FIGS. 10A-10C is a reinforcing device that may employ the features described above regarding the tissue-penetrating elements, comprising outer-edge tines 1005 and center tines 1010. The center tine 1010 may engage tissue during deployment. FIG. 10D illustrates an embodiment of the laser-cut shape of a reinforcing device prior to deployment. As shown in FIGS. 10A-10D, the reinforcing device may be a hollow cylindrical-shaped structure with radial symmetry when flattened, comprising a first end and a second end when not flattened, wherein the second end ends with a penetrating element. This radial symmetry may be further formed in an elliptical geometry that results in a biased force to be applied to the tissue in a particular direction.

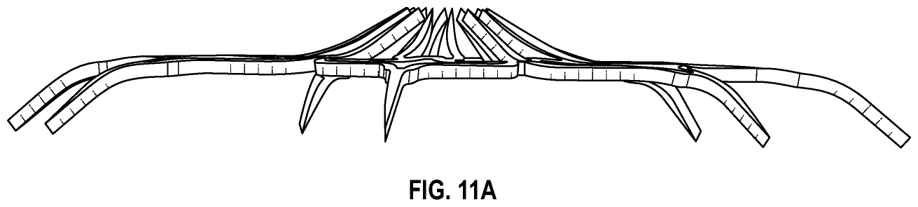

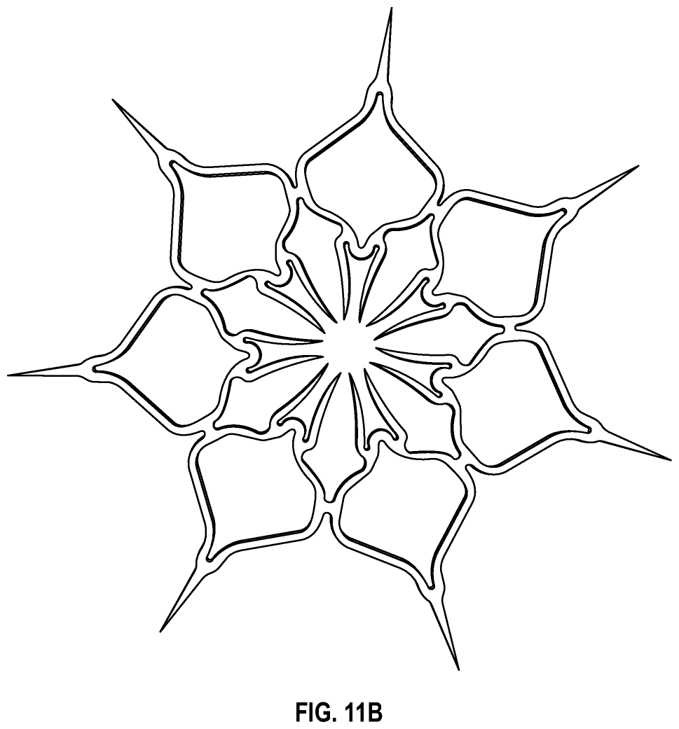

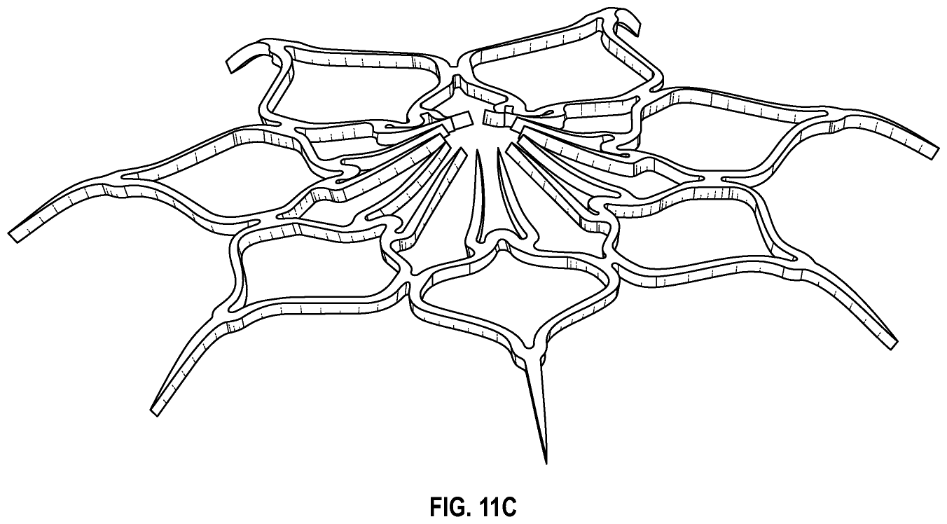

FIG. 11A-11C generally illustrates an embodiment of an annulus repair system as disclosed herein. As shown in FIGS. 11A-11C, a reinforcing device may be circular or somewhat star-shaped, comprising inward-pointing elements designed and configured to engage the tissue and pull inward when deployed/activated. These inward-pointing elements possess features that facilitate the engagement and secure containment of the tissue. In one embodiment, a texture/intentional disruption/pattern may be featured on the cutting edges of the elements to reduce the force it takes for the elements to penetrate the tissue.

FIGS. 12-15 generally illustrates an embodiment of a reinforcing device of an annulus repair system as disclosed herein. As shown in FIGS. 12-15, a reinforcing device may comprise straight penetration elements at each end. This design could also provide a biased force by utilizing only 2, opposing penetrating elements.

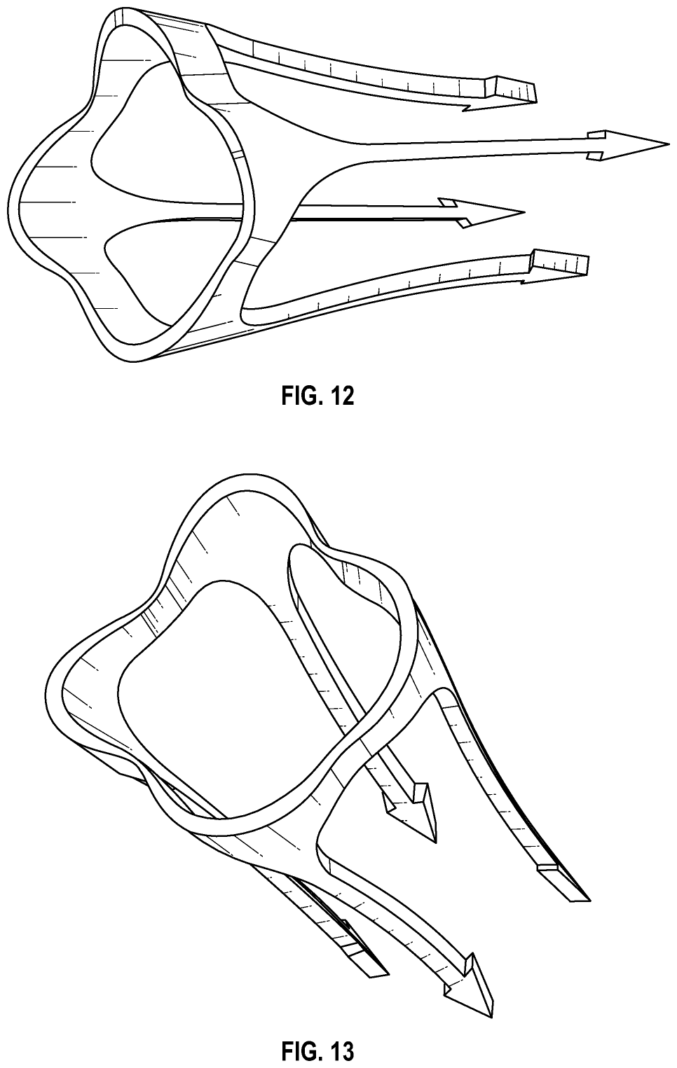

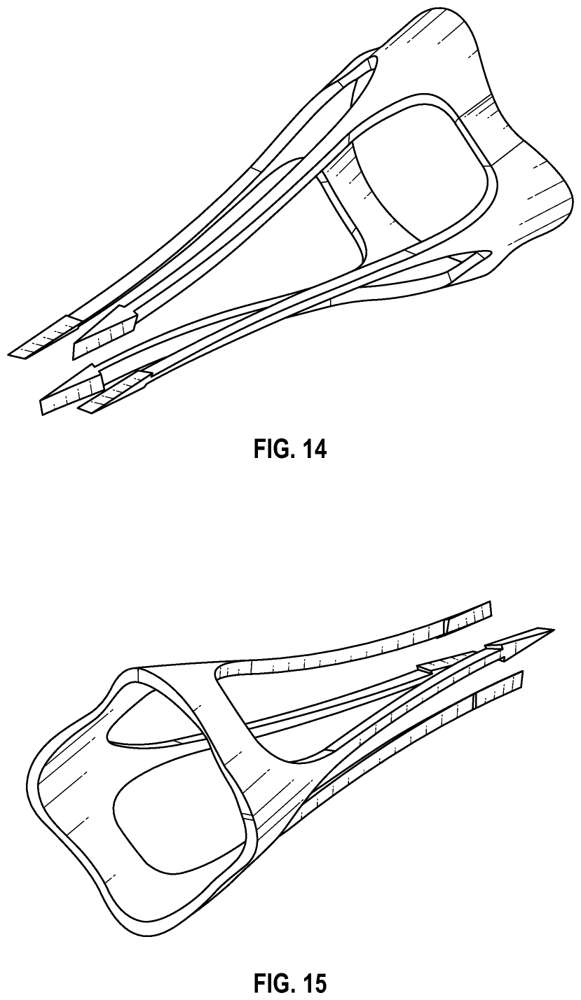

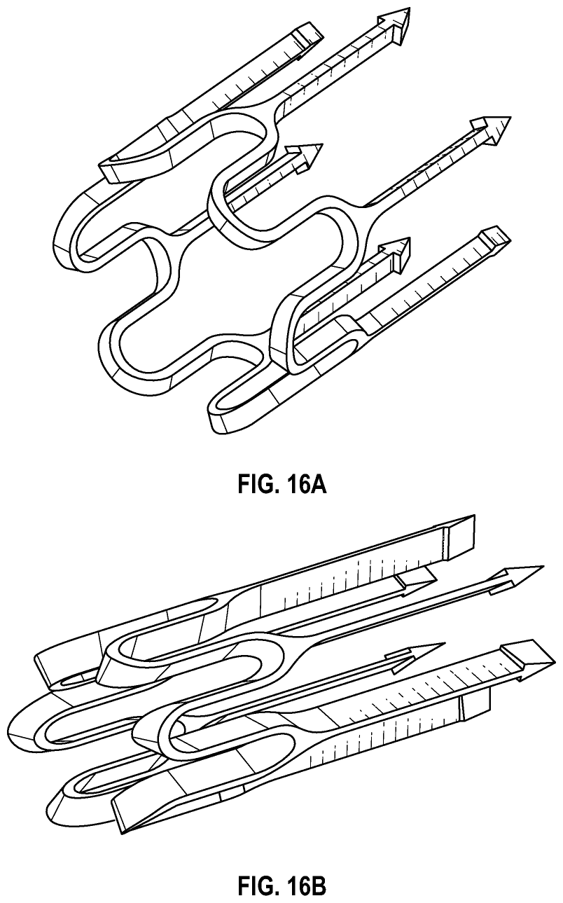

FIGS. 16A-16B generally illustrates an embodiment of a reinforcing device of an annulus repair system as disclosed herein. FIG. 16A illustrates a reinforcing device in the in-catheter configuration, wherein the perimeter of the reinforcing device is s-shaped and comprises barbs and tines. FIG. 16B illustrates the same s-shaped reinforcing device set using circumferential compression. FIG. 16B also illustrates the reinforcing device as deployed into the skin.

FIGS. 17A-17B generally illustrates an embodiment of a reinforcing device of an annulus repair system as disclosed herein. FIG. 17A illustrates a reinforcing device in the in-catheter configuration, wherein the perimeter of the reinforcing device is circular-shaped, comprises barbs and tines, and further comprises strain-relief geometry 1705. FIG. 17B illustrates the same circular-shaped reinforcing device set using circumferential compression. FIG. 17B also illustrates the reinforcing device as deployed into the skin.

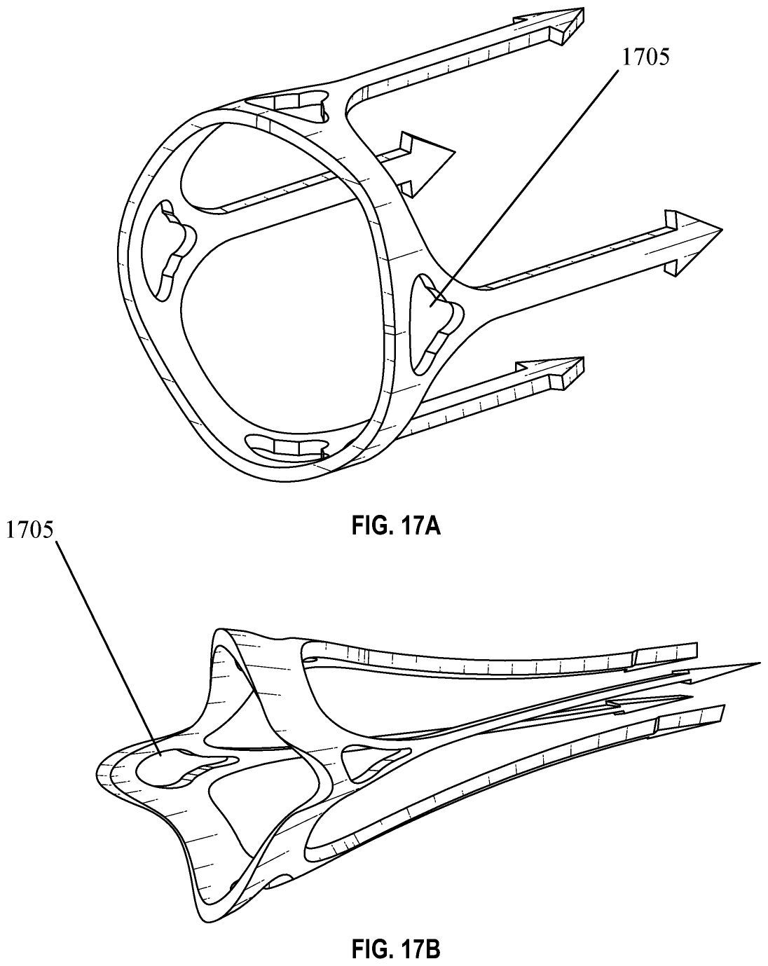

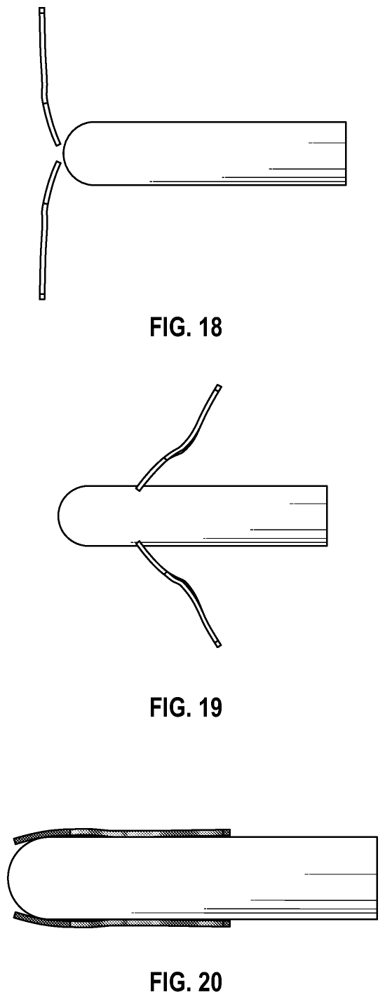

FIGS. 18-20 generally illustrates an embodiment of an annulus repair system as disclosed herein. Specifically, FIGS. 18-19 illustrates the loading shape-set geometry onto the delivery tool. FIG. 20 illustrates the geometry fully loaded onto the delivery tool and ready for deployment.

Still, other embodiments may employ heating mechanisms that do not require a catheter or penetration of the skin or tissue. For example, location of the area to be treated may be determined by 3D-mapping, ultrasound, or other methods. Once the location is determined, electromagnetic heat may be applied to the localized area.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described. One skilled in the art will also appreciate that the s-shaped members may be oriented in a vertical manner (e.g., FIGS. 12-17), or may be oriented in a horizontal manner in a device, with a profile similar to FIGS. 9-11. “Horizontal” and “Vertical” here are shown as clear examples, but it should be evident to one skilled in the art that this orientation may take the form of any angle from vertical to horizontal to an inverted orientation.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

What is claimed is:

1. A heart-valve annulus reinforcing device, comprising:
a hollow cylindrical structure with a top end and a bottom end, wherein both the top end and the bottom end are open;
a plurality of arms extending down from the top end, wherein the plurality of arms end with a penetrating element and wherein the plurality of arms are configured to bend out away from the device.

2. The device of claim 1, wherein the plurality of arms comprises a plurality of elongated slot openings, wherein the plurality of elongated slot openings extends along a portion of the plurality of arms between the top end and the bottom end.

3. The device of claim 1, wherein the penetrating element comprises a rounded blade with a pointed end, wherein the rounded blade is curved inwards towards the center of the cylindrical structure.

4. The device of claim 1, comprising a hinge connection between a first and a second arm of the plurality of arms.

5. A heart-valve annulus reinforcing device, comprising:
a hollow cylindrical-shaped structure with radial symmetry when flattened, comprising a first end and a second end when not flattened;
wherein the second end ends with a penetrating element.

6. The device of claim 5, wherein the first end ends with a penetrating element.

7. The device of claim 5, wherein the first end comprises a circular-opening end.

8. A method for reducing the circumference of an annulus, comprising:
inserting a reinforcing device into a delivery catheter system, wherein the reinforcing device comprises at least one of a heating reinforcer and a clamping reinforcer;
delivering, by the delivery system, the reinforcing device to a tissue; and
deploying the reinforcing device to an area of the tissue by a deployment mechanism of the delivery system, wherein the deployment mechanism is one or both of a coaxial pusher and a pusher platform.

9. The method of claim 8, wherein the clamping reinforcer is a hollow cylindrical structure with a top end and a bottom end, wherein both the top end and the bottom end are open, wherein a plurality of arms extend down from the top end, wherein the plurality of arms end with a penetrating element, and wherein the plurality of arms are configured to bend out away from the device;
wherein inserting a reinforcing device into a delivery catheter system comprises inserting the clamping reinforcer with the plurality of arms bent out; and
wherein deploying the reinforcing device further comprises:
positioning the clamping reinforcer against the tissue at a desired location;
deploying the clamping reinforcer at the desired location of the tissue such that the bent plurality of arms are straightened;
penetrating the tissue with the penetrating element;
grasping a portion of the tissue with the penetrating element;
pulling together the grasped portion of the tissue with the penetrating element, and
releasing the clamping reinforcer from the delivery system.

10. The method of claim 9, wherein the plurality of arms comprises a plurality of elongated slot openings, wherein the plurality of elongated slot openings extends along a portion of the plurality of arms between the top end and the bottom end.

11. The method of claim 9, wherein the penetrating element comprises a rounded blade with a pointed end, wherein the rounded blade is curved inwards towards the center of the cylindrical structure.

12. The method of claim 9, comprising a hinge connection between a first and a second arm of the plurality of arms.

13. The method of claim 9, wherein the penetrating element is coated with a tissue-absorbable chemical composition.

14. The method of claim 8, wherein the clamping reinforcer is a hollow cylindrical-shaped structure with radial symmetry when flattened, comprising a first end and a second end when not flattened, wherein the second end ends with a penetrating element;
wherein deploying the reinforcing device further comprises:
positioning the clamping reinforcer against the tissue at a desired location;
penetrating the tissue with the penetrating element;
grasping a portion of the tissue with the penetrating element;
pulling together the grasped portion of the tissue with the penetrating element, and
releasing the clamping reinforcer from the delivery system.

15. The method of claim 14, wherein the first end ends with a penetrating element.

16. The method of claim 14, wherein the first end comprises a circular-opening end.

17. The method of claim 8, wherein the heating reinforcer is a heat-producing device, wherein deploying the reinforcing device further comprises:
positioning the heating reinforcer against the tissue at a desired location; and
deploying heat to the tissue at the desired location.

18. The method of claim 17, wherein the heat-producing device is one of an electric-heating mechanism, an electromagnetism-heating mechanism, or a laser-heating mechanism.

19. The method of claim 8, further comprising:
retracting the deployment mechanism through a second reinforcing device; and
deploying the second reinforcing device to the area of the tissue by the deployment mechanism of the delivery system after deployment of a first reinforcing device.

20. The method of claim 19, wherein the first-deployed reinforcing device is the clamping reinforcer and the second reinforcing device is the heating reinforcer.
